(19) United States
(12) Patent Application Publication
(10) Pub. No.: US 2017/0128274 A1
(43) Pub. Date: May 11, 2017

(54) Methods and Apparatuses for Registering Substrates in Absorbent Article Converting Lines

(71) Applicant: The Procter & Gamble Company, Cincinnati, OH (US)

(72) Inventors: Stephen Michael Varga, Loveland, OH (US); Taylor Javier Morris, Cincinnati, OH (US)

(21) Appl. No.: 15/344,693

(22) Filed: Nov. 7, 2016

Related U.S. Application Data
(60) Provisional application No. 62/253,710, filed on Nov. 11, 2015.

(51) Int. Cl. A61F 13/15; A61F 13/84

(52) U.S. Cl. CPC A61F 13/15585; A61F 13/15772; A61F 13/84; A61F 2013/1578; A61F 2013/8497

(57) ABSTRACT

The present disclosure relates to methods and apparatuses for detecting registration features. The apparatuses and methods may utilize sensors in combination with cylindrical optics that blurs the image sampling area in the cross direction, while maintaining focus in the machine direction. The blurring may create an averaging or blending effect of the hue values across the sampled area. The sensors may include red, green, blue (RGB) analog outputs that can characterize sensed registration features by a unique sequence that can be compared with a reference sequence. In turn, the substrate speed and/or tension can be adjusted base on the comparison to control the relative placement of advancing substrates and discrete components in converting lines.

### FIELD OF THE INVENTION

The present disclosure relates to systems and methods for manufacturing disposable absorbent articles, and more particularly, systems and methods utilizing cylindrical optics to register advancing substrates in absorbent article converting lines.

### BACKGROUND OF THE INVENTION

Along an assembly line, diapers and various types of other absorbent articles may be assembled by adding components to and otherwise modifying an advancing, continuous web of material. For example, in some processes, advancing webs of material are combined with other advancing webs of material. In other examples, individual components created from advancing webs of material are combined with advancing webs of material, which in turn, are then combined with other advancing webs of material. Once the desired component parts are assembled, the advancing web(s) and component parts are subjected to a final knife cut to separate the web(s) into discrete diapers or other absorbent articles. The discrete diapers or absorbent articles may also then be folded and packaged.

In some manufacturing operations, a continuous base web of material is advanced in a machine direction along a converting line. Discrete components and continuous webs are combined with the base web of material to form a continuous length of absorbent articles. As such, it may be necessary to accurately control the speed and/or tension of the base web to help ensure that the final knife cut is applied at desired locations to help maintain the desired pitch length of the assembled articles. In some manufacturing operations, substrates that are pre-printed with graphics are also converted into various components and are incorporated into absorbent articles. Such graphic designs may be incorporated into absorbent articles to enhance the aesthetic appearance and consumer acceptance. Thus, it may be necessary to control the speeds and/or tension of the pre-printed substrates during manufacture to ensure that the graphics are properly placed in relation to other components of the absorbent articles and/or to ensure the pre-printed substrate is not cut at an undesirable location, such as through a graphic.

As such, manufacturing processes may utilize registration control systems to control the speed and/or tension of advancing substrates. Such registration systems may include various types of sensors adapted to detect various components and/or characteristics of advancing webs to determine if the advancing web is properly positioned with respect to various manufacturing operations, such as cutting and combining operations. Some registration control systems may utilize registration marks printed on a base web to determine if the base web is properly positioned. As the base web advances, the registration marks pass by a sensor that detects the presence of the registration marks. The sensor provides a feedback signal that corresponds to the detection of a registration mark. A controller receives the feedback signal from the sensor and compares the feedback signal with a setpoint. Based on the comparison, the controller may change the speed of the base web.

Some registration control systems may utilize photoelectric sensors that are programmed to detect a specific color of a registration mark. For example, some systems may utilize a color sensor, such as a red, green, blue (RGB) color sensor, that may be calibrated or “trained” to detect a specific registration mark color. As a substrate passes under the sensor, the sensor computes the delta R, G, and B values. A threshold for the detected changes in R, G, and B values may be stored in the sensor, such that changes above the threshold trigger a registration mark detection, and changes below the threshold do not trigger registration mark detection. As such, other printed graphics along the machine direction path between the registration marks (“quiet zones”) may be configured such that the color components of the registration marks are not present.

However, existing systems that are configured to detect the specific colors of registration marks may have certain drawbacks. In particular, the use of registration marks having specific colors may place undesirable limits design choices on other graphics, and current registration mark detection methods may not accommodate for other variations in the manufacturing operations, such as variations in print quality, material properties, and/or web handling operations. Such variable factors may contribute to inconsistent and/or false registration mark detections, and can lead to false product rejects, false web speed adjustments, and discreet unit phasing errors.

In addition, some sensors are configured with spherical lenses that blur the registration marks similarly in both machine and cross directions, which in turn, may impair the ability of the sensor to precisely detect the locations of advancing registration marks. In some configurations, the sensor may not detect the registration marks at consistent spacing due to noise introduced by the surrounding colors on the substrate. As such, registration mark detection may be inconsistent and sometimes may result in missed or false detections. To help reduce missed mark detections, the RGB threshold value stored in the sensor may be set to a relatively low value. However, the relatively low threshold value may also result in increased early, extended, and/or false mark detections. In contrast, raising the RGB threshold to a relatively high value may help the sensor detect registration marks relatively closer to designed positions. However, the relatively high threshold value may also result in increased instances of missed mark detections due to decreased delta RGB values of the combined color profile surrounding certain registration marks. It is to be appreciated that such early, late, and missed mark detections may cause other problems with manufacturing processes. For example, early mark detections may be interpreted by the control system as the base web moving too fast, and late mark detections may be interpreted by the controller as the base web moving too slow. In turn, the controller may erroneously adjust the base web speed if detections are falsely shifted from the intended registration mark position.

Further, absorbent articles manufactured with registration marks included in the final assembly may detract from other pleasing aspects of the absorbent articles. As such, some manufacturing systems are configured such that registration marks are eliminated from the final assembly. For example, some manufacturing systems may be configured to trim the registration marks from the final assembly. However, trimming registrations marks require additional manufacturing operations and equipment. As opposed to trimming the marks from the substrates, other registration systems may utilize sophisticated image detection systems utilizing cameras that are programmed to detect specific shapes of particular objects and/or graphics as opposed to distinct and dedicated registration marks. However, the programming of such imaging systems can be cumbersome as compared to relatively less complex color sensing systems. In addition, the cameras may also provide inconsistent detections as a result of inconsistent shapes of detectable objects and/or graphics. Further, at relatively high base web speeds, time delays within such imaging systems may result in inaccurate registration and control of base web speed. In turn, unstable and/or inaccurate base web speed control may result in misplaced final knife cuts on the continuous length of absorbent articles, resulting in damaged and/or defective absorbent articles.

Consequently, it would be beneficial to provide registration systems and methods that are configured to utilize existing photoelectric color sensors while at the same time providing relatively precise registration mark detection during absorbent article assembly processes and web handling. It would also be beneficial to utilize such systems to enable precise registration control without the need for separate, distinct, dedicated, and/or uniquely colored registration marks.

### SUMMARY OF THE INVENTION

The present disclosure relates to methods and apparatuses for detecting registration features and controlling the relative placement of advancing substrates and discrete components in absorbent article converting lines. The systems and methods herein may utilize sensors in combination with cylindrical optics that blurs the image sampling area appreciably in the cross direction, while maintaining focus in the machine direction. Such blurring may create an averaging or blending effect of the hue values across the sampled area. The sensors may include red, green, blue (RGB) analog outputs that can characterize sensed registration features by a unique sequence that can be compared with a reference sequence. In turn, the substrate speed and/or tension can be adjusted base on the comparison.

In one form, a method for assembling disposable absorbent articles comprises the steps of: providing a continuous substrate extending in a machine direction and defining a width in a cross direction, the continuous substrate comprising a first surface and an opposing second surface, the continuous substrate further comprising registration features; providing a sensor; establishing a detection zone on the first surface of the substrate by positioning a convex cylindrical lens between the first surface of the substrate and the sensor, the convex cylindrical lens comprising a first surface and an opposing convex surface, the convex surface comprising an apex line extending in the machine direction, wherein the first surface of the convex cylindrical lens is in a facing relationship with the first surface of the substrate, and wherein the detection zone defines a length extending the machine direction and width extending in the cross direction such that the detection zone is elongated in the cross direction relative to the machine direction; advancing the registration features through the detection zone by advancing the substrate in the machine direction at a first speed; defocusing light reflected from the detection zone through the convex cylindrical lens; detecting the defocused light passing from the convex cylindrical lens with the sensor; and generating signals corresponding with hue values of detected defocused light reflected from the registration features advancing through the detection zone.

In another form, a method for assembling disposable absorbent articles comprises the steps of: providing a continuous substrate extending in a machine direction and defining a width in a cross direction, the continuous substrate comprising a first surface and an opposing second surface, the continuous substrate further comprising registration features; illuminating an elongate illumination zone extending in the cross direction on the first surface of the advancing substrate; providing a convex cylindrical lens comprising a first surface and an opposing convex surface, the convex surface comprising an apex line extending in the machine direction, wherein the first surface of the convex cylindrical lens is in a facing relationship with the first surface of the advancing substrate; advancing the registration features through the illumination zone by advancing the substrate in the machine direction at a first speed; defocusing light reflected from the elongate illumination zone through the convex cylindrical lens; sensing the defocused light passing from the convex cylindrical lens; and generating signals corresponding with hue values of sensed defocused light reflected from the registration features advancing through the illumination zone.

In yet another form, an absorbent article manufacturing apparatus for controlling the speed of a continuous substrate extending in a machine direction and defining a width in a cross direction, a first surface and an opposing second surface, and registration features, comprises: a convex cylindrical lens comprising a first surface and an opposing convex surface, the convex surface comprising an apex line extending in the machine direction, wherein the first surface of the convex cylindrical lens is in a facing relationship with the first surface of the substrate, and defining a detection zone having a length L extending the machine direction and width W extending in the cross direction, wherein W is greater than L such that the detection zone is elongated in the cross direction relative to the machine direction; a sensor positioned adjacent the convex cylindrical lens to define a detection zone on the first surface of the substrate and having a length L extending the machine direction and width W extending in the cross direction, wherein W is greater than L such that the detection zone is elongated in the cross direction relative to the machine direction, the sensor configured to generate signals corresponding with hue values of detected defocused light from the convex cylindrical lens as reflected from the registration features advancing through the detection zone; and an analyzer selected from the group consisting of: a field programmable gate array, an application specific integrated circuit, and graphical processing unit, the analyzer configured to transform the signals from the sensor into a unique sequence and to adjust the speed of the substrate based on a comparison of the unique sequence to a reference sequence.

### BRIEF DESCRIPTION OF THE DRAWINGS

FIG. 1A is a partially cut away plan view of an absorbent article in the form of a taped diaper that may include one or more substrates monitored and/or controlled in accordance with the present disclosure with the portion of the diaper that faces away from a wearer oriented towards the viewer.

FIG. 1B is a partially cut away plan view of the absorbent article of FIG. 1A that may include one or more substrates monitored and/or controlled in accordance with the present disclosure with the portion of the diaper that faces toward a wearer oriented towards the viewer.

FIG. 1C is a plan view of a diaper with graphics on a backsheet and a connection zone.

FIG. 2A is a front perspective view of an absorbent article in the form of a diaper pant with graphics on a chassis and front and rear belts.

FIG. 2B is a front view of the absorbent article of FIG. 2A.

FIG. 2C is a rear view of the absorbent article of FIG. 2A.

FIG. 3A is a schematic isometric view of a registration system for monitoring and controlling an advancing substrate.

FIG. 3B is a side view of the registration system and advancing substrate taken along the sectional line 3B-3B of FIG. 3A.

FIG. 3C is a side view of the registration system and advancing substrate taken along the sectional line 3C-3C of FIG. 3B.

FIG. 3D is a schematic view of the registration system and advancing substrate taken along the sectional line 3D-3D of FIG. 3A.

FIG. 3E is a schematic view of the registration system and advancing substrate taken along the sectional line 3E-3E of FIG. 3D.

FIG. 4 is a schematic isometric view of a registration system for monitoring and controlling an advancing substrate including an illumination apparatus.

FIG. 5A is a schematic view of the registration system monitoring and controlling an advancing substrate with identical graphics positioned along the machine direction, wherein the graphics are used as registration features.

FIG. 5B is a schematic view of the registration system and advancing substrate taken along the sectional line 5B-5B of FIG. 5A.

FIG. 5C is a schematic view of the registration system monitoring and controlling an advancing substrate with graphics and dedicated registration marks positioned along the machine direction.

FIG. 6A is a schematic view of the registration system monitoring and controlling an advancing substrate with different graphics positioned along the machine direction, wherein the graphics are used as registration features.

FIG. 6B is a schematic view of the registration system and advancing substrate taken along the sectional line 6B-6B of FIG. 6A.

FIG. 7 is a schematic view of the registration system monitoring and controlling an advancing substrate with graphics positioned along the machine direction, wherein the graphics are used as registration features.

### DETAILED DESCRIPTION OF THE INVENTION

The following term explanations may be useful in understanding the present disclosure:

“Absorbent article” is used herein to refer to consumer products whose primary function is to absorb and retain soils and wastes. “Diaper” is used herein to refer to an absorbent article generally worn by infants and incontinent persons about the lower torso. The term “disposable” is used herein to describe absorbent articles which generally are not intended to be laundered or otherwise restored or reused as an absorbent article (e.g., they are intended to be discarded after a single use and may also be configured to be recycled, composted or otherwise disposed of in an environmentally compatible manner).

The term “taped diaper” (also referred to as “open diaper”) refers to disposable absorbent articles having an initial front waist region and an initial back waist region that are not fastened, pre-fastened, or connected to each other as packaged, prior to being applied to the wearer. A taped diaper may be folded about the lateral centerline with the interior of one waist region in surface to surface contact with the interior of the opposing waist region without fastening or joining the waist regions together. Example taped diapers are disclosed in various suitable configurations U.S. Pat. Nos. 5,167,897, 5,360,420, 5,599,335, 5,643,588, 5,674,216, 5,702,551, 5,968,025, 6,107,537, 6,118,041, 6,153,209, 6,410,129, 6,426,444, 6,586,652, 6,627,787, 6,617,016, 6,825,393, and 6,861,571; and U.S. Patent Publication Nos. 2013/0072887 A1; 2013/0211356 A1; and 2013/0306226 A1.

The term “pant” (also referred to as “training pant”, “pre-closed diaper”, “diaper pant”, “pant diaper”, and “pull-on diaper”) refers herein to disposable absorbent articles having a continuous perimeter waist opening and continuous perimeter leg openings designed for infant or adult wearers. A pant can be configured with a continuous or closed waist opening and at least one continuous, closed, leg opening prior to the article being applied to the wearer. A pant can be preformed or pre-fastened by various techniques including, but not limited to, joining together portions of the article using any refastenable and/or permanent closure member (e.g., seams, heat bonds, pressure welds, adhesives, cohesive bonds, mechanical fasteners, etc.). A pant can be preformed anywhere along the circumference of the article in the waist region (e.g., side fastened or seamed, front waist fastened or seamed, rear waist fastened or seamed). Example diaper pants in various configurations are disclosed in U.S. Pat. Nos. 5,246,433; 5,569,234; 6,120,487; 6,120,489; 4,940,464; 5,092,861; 5,897,545; 5,957,908; and U.S. Patent Publication No. 2003/0233082.

An “elastic,” “elastomer” or “elastomeric” refers to materials exhibiting elastic properties, which include any material that upon application of a force to its relaxed, initial length can stretch or elongate to an elongated length more than 10% greater than its initial length and will substantially recover back to about its initial length upon release of the applied force.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

As used herein, the terms “registration process,” “registration system,” “registration,” or “registering” refer to a machine control process or system for controlling a substrate, (which can have multiplicity of pre-produced objects, such as graphics, spaced on the substrate at a pitch interval that may vary in the machine direction) through a converting line producing articles, by providing a positional adjustment of the pre-produced objects on the substrate to a target position constant associated with a pitched unit operation of the converting line.

As used herein, the term “graphic” refers to images or designs that are constituted by a figure (e.g., a line(s)), a symbol or character, a color difference or transition of at least two colors, or the like. A graphic may include an aesthetic image or design that can provide certain benefit(s) when viewed. A graphic may be in the form of a photographic image. A graphic may also be in the form of a 1-dimensional (1-D) or 2-dimensional (2-D) bar code or a quick response (QR) bar code. A graphic design is determined by, for example, the color(s) used in the graphic (individual pure ink or spot colors as well as built process colors), the sizes of the entire graphic (or components of the graphic), the positions of the graphic (or components of the graphic), the movements of the graphic (or components of the graphic), the geometrical shapes of the graphic (or components of the graphics), the number of colors in the graphic, the variations of the color combinations in the graphic, the number of graphics printed, the disappearance of color(s) in the graphic, and the contents of text messages in the graphic.

As used herein, the term “registration feature” refers to a signaling mechanism that is recognizable by a machine. For example, registration features may be in the form of printed graphics on a substrate and/or components. Registration features may be in the form of separately printed graphics and may have a unique color, such as printed rectangular-shaped marks. In some configurations, all or portions of graphics on a substrate and/or components may be composed of registration features. In some examples, registration features may be in the form of physical discontinuities such as notches, protrusions, depressions, or holes formed in a substrate and/or components. Registration features provide optical markers that operate on the basis of providing detectable changes in intensities of visible and/or non-visible wavelengths of light.

“Longitudinal” means a direction running substantially perpendicular from a waist edge to a longitudinally opposing waist edge of an absorbent article when the article is in a flat out, uncontracted state, or from a waist edge to the bottom of the crotch, i.e. the fold line, in a bi-folded article. Directions within 45 degrees of the longitudinal direction are considered to be “longitudinal.” “Lateral” refers to a direction running from a longitudinally extending side edge to a laterally opposing longitudinally extending side edge of an article and generally at a right angle to the longitudinal direction. Directions within 45 degrees of the lateral direction are considered to be “lateral.”

The term “substrate” is used herein to describe a material which is primarily two-dimensional (i.e. in an XY plane) and whose thickness (in a Z direction) is relatively small (i.e. 1/10 or less) in comparison to its length (in an X direction) and width (in a Y direction). Non-limiting examples of substrates include a web, layer or layers or fibrous materials, nonwovens, films and foils such as polymeric films or metallic foils. These materials may be used alone or may comprise two or more layers laminated together. As such, a web is a substrate.

The term “nonwoven” refers herein to a material made from continuous (long) filaments (fibers) and/or discontinuous (short) filaments (fibers) by processes such as spunbonding, meltblowing, carding, and the like. Nonwovens do not have a woven or knitted filament pattern.

The term “machine direction” (MD) is used herein to refer to the direction of material flow through a process. In addition, relative placement and movement of material can be described as flowing in the machine direction through a process from upstream in the process to downstream in the process.

The term “cross direction” (CD) is used herein to refer to a direction that is generally perpendicular to the machine direction.

The present disclosure relates to methods and apparatuses for assembling absorbent articles, and in particular, to registration systems and methods for detecting registration features and controlling the relative placement of advancing substrates and discrete components in diaper converting lines. More particularly, the systems and methods herein may utilize sensors in combination with cylindrical optics. The sensors may include red, green, blue (RGB) analog outputs that can characterize sensed registration features, such as colors of printed graphics, on advancing substrates by a unique sequence across a specific channel or a combination of channels. The unique sequence can then be compared with a reference sequence. In some configurations, the RGB channels may be transformed into alternative orthogonal spaces such as HSL (hue, saturation, luminance) in order to improve the differentiation of the acquired sequence with respect to the reference sequence. In turn, the substrate speed and/or tension can be adjusted base on the comparison. Utilizing cylindrical optics, in contrast to the spherical optics used in existing systems, blurs the image sampling area appreciably in the cross direction, while maintaining focus in the machine direction. Such blurring may create an averaging or blending effect of the hue values across the sampled area, thus mitigating and/or eliminating the negative consequences of inconsistent and/or false detections resulting from variations in print quality, material properties (such as cross directional width), and/or web handling operations (such as substrate mistracking). In turn, the unique sequence corresponding with a registration feature may be in the form of a waveform defined by sensed averaged or blurred hue values of the registration feature along a machine direction of an advancing substrate. As such, the use of a single point, multi-channel color sensor combined with cylindrical optics that sample across a segment of a registration feature, such as a printed graphic, may help increase design flexibility, achieve robust registration outputs, and eliminate the need for complex imaging systems, dedicated, distinct registration marks, and/or quiet zones.

As discussed below, the systems and methods herein utilize a sensor and a convex cylindrical lens to detect registration features on an advancing substrate extending in a machine direction and defining a width in a cross direction. The convex cylindrical lens includes a first surface and an opposing convex surface and is positioned between the substrate and the sensor. The convex surface includes an apex line extending in the machine direction, wherein the first surface of the convex cylindrical lens is in a facing relationship with the first surface of the advancing substrate. The relative positions of the sensor, the convex cylindrical lens, and the substrate establishes a detection zone on the substrate. The detection zone defines a length L extending the machine direction and width W extending in the cross direction, wherein W is greater than L such that the detection zone is elongated in the cross direction relative to the machine direction. The registration features are advanced through the detection zone by advancing the substrate in the machine direction, and light is reflected from the elongate detection zone through the convex cylindrical lens. The reflected light is defocused or blurred by the cylindrical lens, and the sensor detects the defocused light passing from the convex cylindrical lens. In turn, the sensor generates signals corresponding with hue values of sensed defocused light reflected from the registration features advancing through the detection zone. The signals may then be transformed into a unique sequence based on a color pattern of the registration feature extending along the machine direction of the substrate. As such, the speed and/or tension of the advancing substrate may be changed based on a comparison of the unique with a reference sequence.

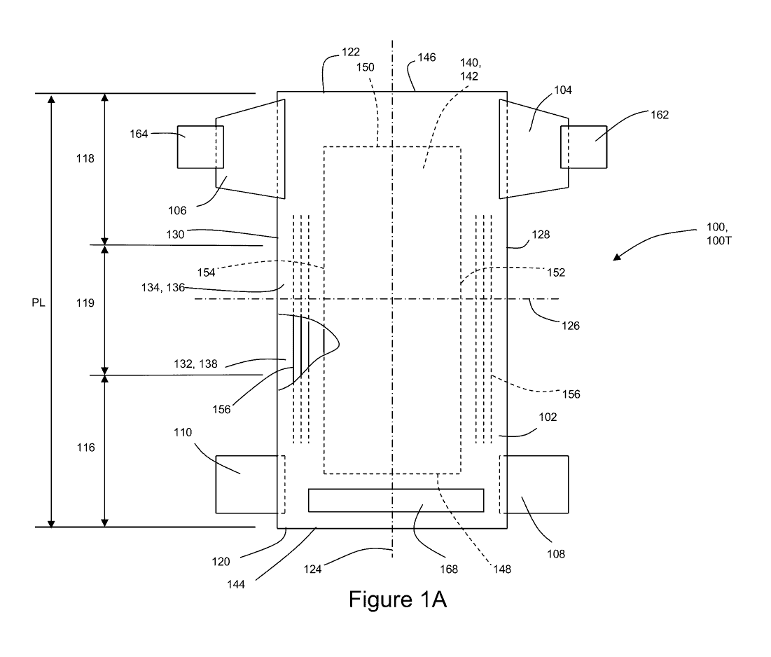

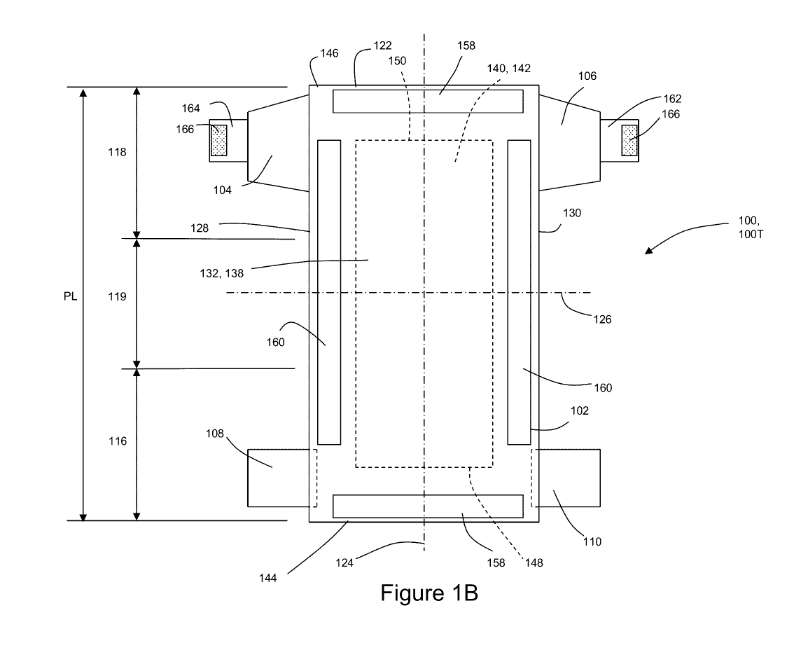

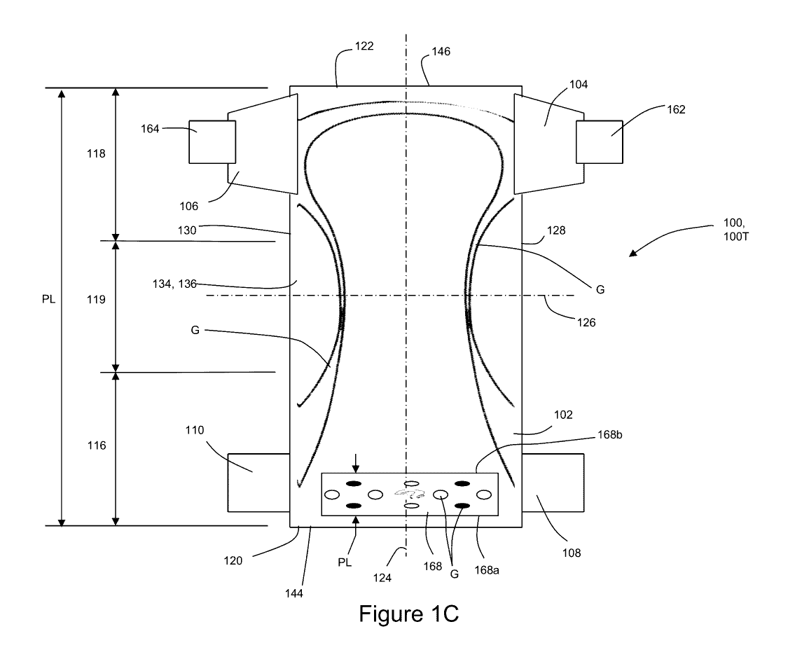

It is to be appreciated that the systems and methods disclosed herein are applicable to work with various types of converting processes and/or machines, such as for example, absorbent article manufacturing, packaging, and/or printing processes. The methods and apparatuses are discussed below in the context of manufacturing diapers. And for the purposes of a specific illustration, FIGS. 1A and 1B show an example of an absorbent article 100 that may be assembled in accordance with the methods and apparatuses disclosed herein. In particular, FIG. 1A shows one example of a plan view of an absorbent article 100 configured as a taped diaper 100T, with the portion of the diaper that faces away from a wearer oriented towards the viewer. And FIG. 1B shows a plan view of the diaper 100 with the portion of the diaper that faces toward a wearer oriented towards the viewer. The taped diaper 100T shown in FIGS. 1A and 1B includes a chassis 102, first and second rear side panels 104 and 106; and first and second front side panels 108 and 110.

As shown in FIGS. 1A and 1B, the diaper 100 and the chassis 102 each include a first waist region 116, a second waist region 118, and a crotch region 119 disposed intermediate the first and second waist regions. The first waist region 116 may be configured as a front waist region, and the second waist region 118 may be configured as back waist region. In some embodiments, the length of each of the front waist region, back waist region, and crotch region may be ⅓ of the length of the absorbent article 100. The absorbent article may also include a laterally extending front waist edge 120 in the front waist region 116 and a longitudinally opposing and laterally extending back waist edge 122 in the back waist region 118. To provide a frame of reference for the present discussion, the diaper 100T in FIGS. 1A and 1B is shown with a longitudinal axis 124 and a lateral axis 126. The longitudinal axis 124 may extend through a midpoint of the front waist edge 120 and through a midpoint of the back waist edge 122. And the lateral axis 126 may extend through a midpoint of a first longitudinal or right side edge 128 and through a midpoint of a second longitudinal or left side edge 130.

As shown in FIGS. 1A and 1B, the diaper 100 includes an inner, body facing surface 132, and an outer, garment facing surface 134. And the chassis 102 may include a backsheet 136 and a topsheet 138. The chassis 102 may also include an absorbent assembly 140, including an absorbent core 142, disposed between a portion of the topsheet 138 and the backsheet 136. As discussed in more detail below, the diaper 100 may also include other features, such as leg elastics and/or leg cuffs, an elastic waist region, and/or flaps, e.g., side panels and/or ears, to enhance the fits around the legs and waist of the wearer, to enhance the fit around the legs of the wearer.

As shown in FIGS. 1A and 1B, the periphery of the chassis 102 may be defined by the first longitudinal side edge 128, a second longitudinal side edge 130, a first laterally extending end edge 144 disposed in the first waist region 116, and a second laterally extending end edge 146 disposed in the second waist region 118. Both side edges 128 and 130 extend longitudinally between the first end edge 144 and the second end edge 146. As shown in FIG. 1A, the laterally extending end edges 144 and 146 may form a portion of the laterally extending front waist edge 120 in the front waist region 116 and a portion of the longitudinally opposing and laterally extending back waist edge 122 in the back waist region 118. The distance between the first lateral end edge 144 and the second lateral end edge 146 may define a pitch length, PL, of the chassis 102. When the diaper 100 is worn on the lower torso of a wearer, the front waist edge 120 and the back waist edge 122 may encircle a portion of the waist of the wearer. At the same time, the side edges 128 and 130 may encircle at least a portion of the legs of the wearer. And the crotch region 119 may be generally positioned between the legs of the wearer with the absorbent core 142 extending from the front waist region 116 through the crotch region 119 to the back waist region 118.

It is to also be appreciated that a portion or the whole of the diaper 100 may also be made laterally extensible. The additional extensibility may help allow the diaper 100 to conform to the body of a wearer during movement by the wearer. The additional extensibility may also help, for example, the user of the diaper 100, including a chassis 102 having a particular size before extension, to extend the front waist region 116, the back waist region 118, or both waist regions of the diaper 100 and/or chassis 102 to provide additional body coverage for wearers of differing size, i.e., to tailor the diaper to an individual wearer. Such extension of the waist region or regions may give the absorbent article a generally hourglass shape, so long as the crotch region is extended to a relatively lesser degree than the waist region or regions, and may impart a tailored appearance to the article when it is worn.

As previously mentioned, the diaper 100 may include a backsheet 136. The backsheet 136 may also define the outer surface 134 of the chassis 102. The backsheet 136 may be impervious to fluids (e.g., menses, urine, and/or runny feces) and may be manufactured in part from a thin plastic film, although other flexible liquid impervious materials may also be used. The backsheet 136 may prevent the exudates absorbed and contained in the absorbent core from wetting articles which contact the diaper 100, such as bedsheets, pajamas and undergarments. The backsheet 136 may also comprise a woven or nonwoven material, polymeric films such as thermoplastic films of polyethylene or polypropylene, and/or a multi-layer or composite materials comprising a film and a nonwoven material (e.g., having an inner film layer and an outer nonwoven layer). The backsheet may also comprise an elastomeric film. An example backsheet 136 may be a polyethylene film having a thickness of from about 0.012 mm (0.5 mils) to about 0.051 mm (2.0 mils). Exemplary polyethylene films are manufactured by Clopay Corporation of Cincinnati, Ohio, under the designation BR-120 and BR-121 and by Tredegar Film Products of Terre Haute, Ind., under the designation XP-39385. The backsheet 136 may also be embossed and/or matte-finished to provide a more clothlike appearance. Further, the backsheet 136 may permit vapors to escape from the absorbent core (i.e., the backsheet is breathable) while still preventing exudates from passing through the backsheet 136. The size of the backsheet 136 may be dictated by the size of the absorbent core 142 and/or particular configuration or size of the diaper 100.

Also described above, the diaper 100 may include a topsheet 138. The topsheet 138 may also define all or part of the inner surface 132 of the chassis 102. The topsheet 138 may be compliant, soft feeling, and non-irritating to the wearer's skin. It may be elastically stretchable in one or two directions. Further, the topsheet 138 may be liquid pervious, permitting liquids (e.g., menses, urine, and/or runny feces) to penetrate through its thickness. A topsheet 138 may be manufactured from a wide range of materials such as woven and nonwoven materials; apertured or hydroformed thermoplastic films; apertured nonwovens, porous foams; reticulated foams; reticulated thermoplastic films; and thermoplastic scrims. Woven and nonwoven materials may comprise natural fibers such as wood or cotton fibers; synthetic fibers such as polyester, polypropylene, or polyethylene fibers; or combinations thereof. If the topsheet 138 includes fibers, the fibers may be spunbond, carded, wet-laid, meltblown, hydroentangled, or otherwise processed as is known in the art.

Topsheets 138 may be selected from high loft nonwoven topsheets, apertured film topsheets and apertured nonwoven topsheets. Apertured film topsheets may be pervious to bodily exudates, yet substantially non-absorbent, and have a reduced tendency to allow fluids to pass back through and rewet the wearer's skin. Exemplary apertured films may include those described in U.S. Pat. Nos. 5,628,097; 5,916,661; 6,545,197; and 6,107,539.

As mentioned above, the diaper 100 may also include an absorbent assembly 140 that is joined to the chassis 102. As shown in FIGS. 1A and 1B, the absorbent assembly 140 may have a laterally extending front edge 148 in the front waist region 116 and may have a longitudinally opposing and laterally extending back edge 150 in the back waist region 118. The absorbent assembly may have a longitudinally extending right side edge 152 and may have a laterally opposing and longitudinally extending left side edge 154, both absorbent assembly side edges 152 and 154 may extend longitudinally between the front edge 148 and the back edge 150. The absorbent assembly 140 may additionally include one or more absorbent cores 142 or absorbent core layers. The absorbent core 142 may be at least partially disposed between the topsheet 138 and the backsheet 136 and may be formed in various sizes and shapes that are compatible with the diaper. Exemplary absorbent structures for use as the absorbent core of the present disclosure are described in U.S. Pat. Nos. 4,610,678; 4,673,402; 4,888,231; and 4,834,735.

Some absorbent core embodiments may comprise fluid storage cores that contain reduced amounts of cellulosic airfelt material. For instance, such cores may comprise less than about 40%, 30%, 20%, 10%, 5%, or even 1% of cellulosic airfelt material. Such a core may comprises primarily absorbent gelling material in amounts of at least about 60%, 70%, 80%, 85%, 90%, 95%, or even about 100%, where the remainder of the core comprises a microfiber glue (if applicable). Such cores, microfiber glues, and absorbent gelling materials are described in U.S. Pat. Nos. 5,599,335; 5,562,646; 5,669,894; and 6,790,798 as well as U.S. Patent Publication Nos. 2004/0158212 and 2004/0097895.

As previously mentioned, the diaper 100 may also include elasticized leg cuffs 156 and an elasticized waistband 158. It is to be appreciated that the leg cuffs 156 can be and are sometimes also referred to as leg bands, side flaps, barrier cuffs, elastic cuffs or gasketing cuffs. The elasticized leg cuffs 156 may be configured in various ways to help reduce the leakage of body exudates in the leg regions. Example leg cuffs 156 may include those described in U.S. Pat. Nos. 3,860,003; 4,909,803; 4,695,278; 4,795,454; 4,704,115; and U.S. Patent Publication No. 2009/0312730 A1.

The elasticized waistband 158 may provide improved fit and containment and may be a portion or zone of the diaper 100 that may elastically expand and contract to dynamically fit a wearer's waist. The elasticized waistband 158 may extend longitudinally inwardly from the waist edges 120, 122 of the diaper toward the lateral edges 148, 150 of the absorbent core 142. The diaper 100 may also include more than one elasticized waistband 158, for example, having one waistband 158 positioned in the back waist region 118 and one waistband 158 positioned in the front wait region 116, although other embodiments may be constructed with a single elasticized waistband 158. The elasticized waistband 158 may be constructed in a number of different configurations including those described in U.S. Pat. Nos. 4,515,595 and 5,151,092. In some embodiments, the elasticized waistbands 158 may include materials that have been “prestrained” or “mechanically prestrained” (subjected to some degree of localized pattern mechanical stretching to permanently elongate the material). The materials may be prestrained using deep embossing techniques as are known in the art. In some embodiments, the materials may be prestrained by directing the material through an incremental mechanical stretching system as described in U.S. Pat. No. 5,330,458. The materials are then allowed to return to their substantially untensioned condition, thus forming a zero strain stretch material that is extensible, at least up to the point of initial stretching. Examples of zero strain materials are disclosed in U.S. Pat. Nos. 2,075,189; 3,025,199; 4,107,364; 4,209,563; 4,834,741; and 5,151,092.

As shown in FIG. 1B, the chassis 102 may include longitudinally extending and laterally opposing side flaps 160 that are disposed on the interior surface 132 of the chassis 102 that faces inwardly toward the wearer and contacts the wearer. Each side flap may have a proximal edge. The side flaps may also overlap the absorbent assembly 140, wherein the proximal edges extend laterally inward of the respective side edges of the absorbent assembly 152 and 154. In some configurations, the side flaps may not overlap the absorbent assembly. It is to be appreciated that the side flaps may be formed in various ways, such as for example, by folding portions of the chassis 102 laterally inward, i.e., toward the longitudinal axis 124, to form both the respective side flaps and the side edges 128 and 130 of the chassis 102. In another example, the side flaps may be formed by attaching an additional layer or layers to the chassis at or adjacent to each of the respective side edges and of the chassis. Each of the side flaps may be joined to the interior surface 132 of the chassis and/or the absorbent assembly in side flap attachment zones in the front waist region 116 and in side flap attachment zones in the back waist region 118. The side flaps may extend to the same longitudinal extent as the absorbent article or alternatively the side flaps may have a longitudinal extent that is less than the absorbent article.

Taped diapers may be manufactured and provided to consumers in a configuration wherein the front waist region and the back waist region are not fastened, pre-fastened, or connected to each other as packaged, prior to being applied to the wearer. For example, the taped diaper 100 may be folded about a lateral centerline with the interior surface 132 of the first waist region 116 in surface to surface contact with the interior surface 132 of the second waist region 118 without fastening or joining the waist regions together. The rear side panels 104 and 106 and/or the front side panels 108 and 110 may also be folded laterally inward toward the inner surfaces 132 of the waist regions 116 and 118.

The diaper 100 may also include various configurations of fastening elements to enable fastening of the front waist region 116 and the back waist region 118 together to form a closed waist circumference and leg openings once the diaper is positioned on a wearer. For example, as shown in FIGS. 1A and 1B, the diaper 100 may include first and second fastening members 162, 164, also referred to as tabs, connected with the first and second rear side panels 104, 106, respectively. The diaper may also include first and second front side panels 108, 110, that may or may not include fastening members.

With continued reference to FIGS. 1A and 1B, each side panel 104, 106 and/or fastening member 162 and 164 may form a portion of or may be permanently bonded, adhered or otherwise joined directly or indirectly to the chassis 102 laterally inward from the side edge 128 and 130, in one of the front waist region 116 or the back waist region 118. Alternatively, the fastening members 162, 164 may form a portion of or may be permanently bonded, adhered or otherwise joined directly or indirectly to the first and second rear panels 104, 106 at or adjacent the distal edge of the panel and/or the first and second front side panels 108 and 110 at or adjacent the distal edge of the side panel. It is to be appreciated that the fastening members and/or side panels may be assembled in various ways, such as disclosed for example, in U.S. Pat. No. 7,371,302. The fastening members 162, 164 and/or side panels 104, 106, 108, 110 may also be permanently bonded or joined at or adjacent the side edges 128 and 130 of the chassis 102 in various ways, such as for example, by adhesive bonds, sonic bonds, pressure bonds, thermal bonds or combinations thereof, such as disclosed for example, U.S. Pat. No. 5,702,551.

Referring now to FIG. 1B, the first fastening member 162 and/or the second fastening member 164 may include various types of releasably engageable fasteners. The first and second fastening members 162 and/or 164 may also include various types of refastenable fastening structures. For example, the first and second fastening members 162 and 164 may include mechanical fasteners, 166, in the form of hook and loop fasteners, hook and hook fasteners, macrofasteners, buttons, snaps, tab and slot fasteners, tape fasteners, adhesive fasteners, cohesive fasteners, magnetic fasteners, hermaphrodidic fasteners, and the like. Some examples of fastening systems and/or fastening members 162, 164 are discussed in U.S. Pat. Nos. 3,848,594; 4,662,875; 4,846,815; 4,894,060; 4,946,527; 5,151,092; 5,221,274; 6,251,097; 6,669,618; 6,432,098; and U.S. Patent Publication Nos. 2007/0078427 and 2007/0093769.

As previously mentioned, the fastening members 162 and 164 may be constructed from various materials and may be constructed as a laminate structure. The fastening members 162 and 164 may also be adapted to releasably and/or refastenably engage or connect with another portion of the diaper 100. For example, as shown in FIG. 1A, the diaper 100 may include a connection zone 168, sometimes referred to as a landing zone, in the first waist region 116. As such, when the taped diaper 100 is placed on a wearer, the fastening members 162 and 164 may be pulled around the waist of the wearer and connected with the connection zone 168 in the first waist region 116 to form a closed waist circumference and a pair of laterally opposing leg openings. It is to be appreciated that the connection zone may be constructed from a separate substrate that is connected with the chassis 102 of the taped diaper, such as shown in FIG. 1C. As such, the connection zone 168 may have a pitch length PL defined by a distance extending between a first lateral end edge 168a and the second lateral end edge 168b. In some embodiments, the connection zone may be integrally formed as part of the backsheet 136 of the diaper 100 or may be formed as part of the first and second front panels 108, 110, such as described in U.S. Pat. Nos. 5,735,840 and 5,928,212.

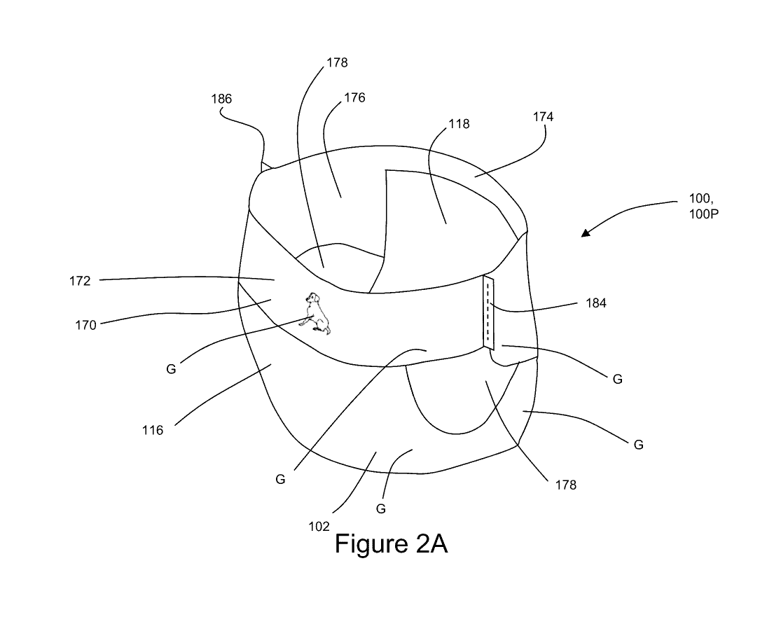

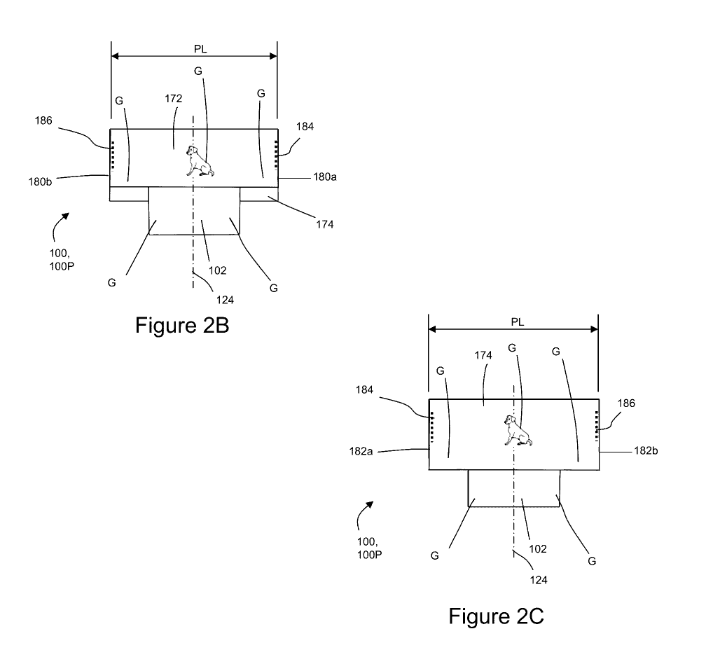

As previously mentioned, absorbent articles 100 may also be configured as diaper pants 100P having a continuous perimeter waist opening and continuous perimeter leg openings. For example, FIG. 2A shows a perspective view of an absorbent article 100 in the form of a diaper pant 100P in a pre-fastened configuration, and FIGS. 2B-2C show front and rear plan views of the diaper pant 100P. The diaper pant 100P may include a chassis 102 such a discussed above with reference to FIG. 1A and a ring-like elastic belt 170 such as shown in FIG. 2A. In some embodiments, a first elastic belt 172 and a second elastic belt 174 are bonded together to form the ring-like elastic belt 170. As such, diaper pants may be manufactured with the ring-like elastic belt 174 and provided to consumers in a configuration wherein the front waist region 116 and the back waist region 118 of the chassis 102 are connected to each other as packaged, prior to being applied to the wearer. As such, diaper pants may have a continuous perimeter waist opening 176 and continuous perimeter leg openings 178 such as shown in FIG. 2A.

As previously mentioned, the ring-like elastic belt 170 may be defined by a first elastic belt 172 connected with a second elastic belt 174. As shown in FIGS. 2A-2C, the first elastic belt 172 extends between a first longitudinal side edge 180a and a second longitudinal side edge 180b. And the second elastic 174 belt extends between a first longitudinal side edge 182a and a second longitudinal side edge 182b. The distance between the first longitudinal side edge 180a and the second longitudinal side edge 180b defines a pitch length, PL, of the first elastic belt 172, and the distance between the first longitudinal side edge 182a and the second longitudinal side edge 182b defines the pitch length, PL, of the second elastic belt 174. The first elastic belt is connected with the first waist region 116 of the chassis 102, and the second elastic belt 108 is connected with the second waist region 116 of the chassis 102. As shown in FIGS. 2A-2C, opposing end regions of the first elastic belt 172 are connected with opposing end regions of the second elastic belt 174 at a first side seam 184 and a second side seam 186 to define the ring-like elastic belt 170 as well as the waist opening 176 and leg openings 178. It is to be appreciated that the ring-like elastic belt may be formed by joining a first elastic belt to a second elastic belt with permanent side seams or with openable and reclosable fastening systems disposed at or adjacent the laterally opposing sides of the belts.

As previously mentioned, absorbent articles may be assembled with various components that require registration control during assembly. It is to be appreciated that absorbent articles herein may include graphics various components. Thus, in the context of the previous discussion, the apparatuses and methods herein may be used to provide for registration of substrates and components during the manufacture of an absorbent article 100. For example, the apparatuses and methods herein may be utilized in registering graphics applied to any of the topsheet 138; backsheet 136; absorbent core 140; leg cuffs 156; waist feature 158; side panels 104, 106, 108, 110; connection zones 168; fastening elements 162, 166, and/or belts during the manufacture of an absorbent article 100. For example, the backsheet 136 of the taped diaper 100T shown in FIG. 1C includes graphics G that may require registration control during assembly. The connection zone 168 shown in FIG. 1C may also include graphics G requiring registration control during assembly. In yet another example, the front belt 172 and rear belt 174 of the diaper pant 100P may include graphics G requiring registration control during assembly. As discussed in more detail below, the systems and methods herein may utilize such graphics as registration features during assembly.

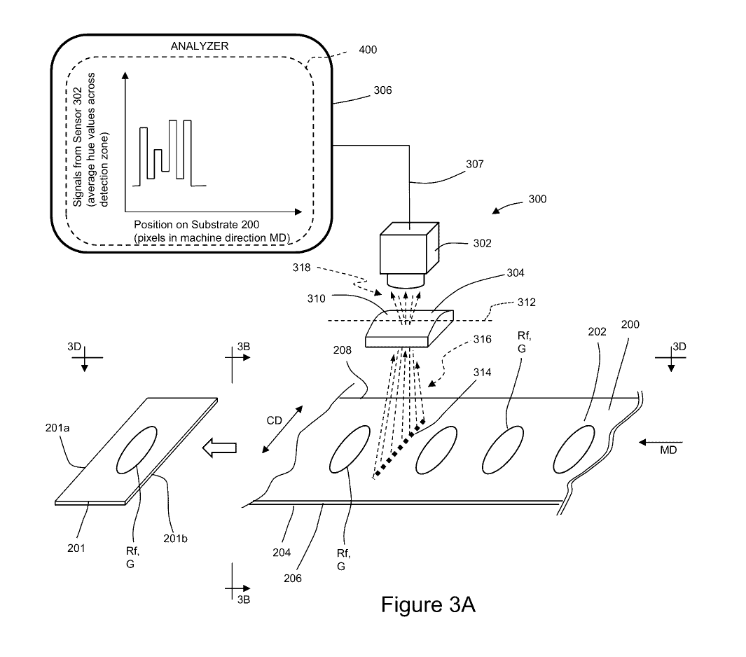

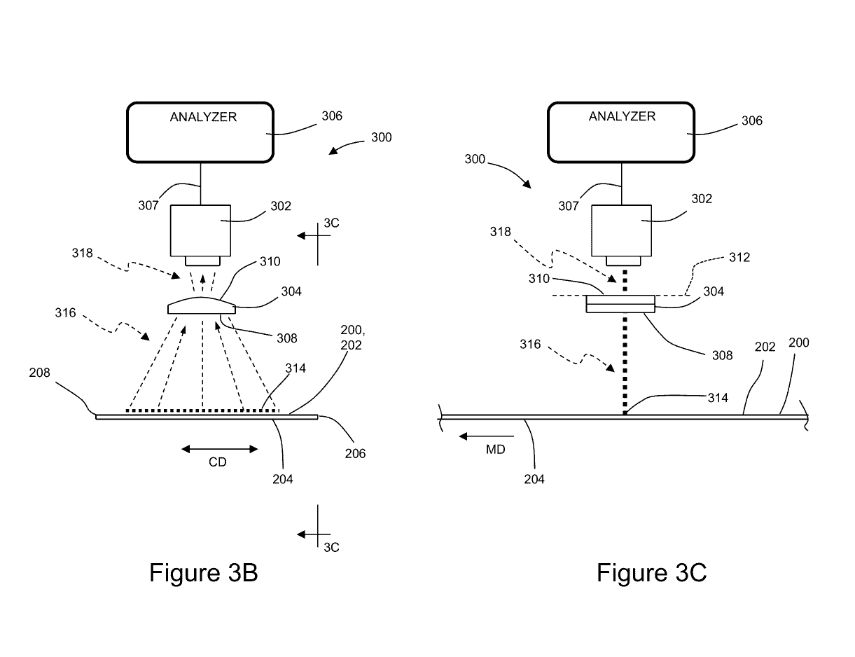

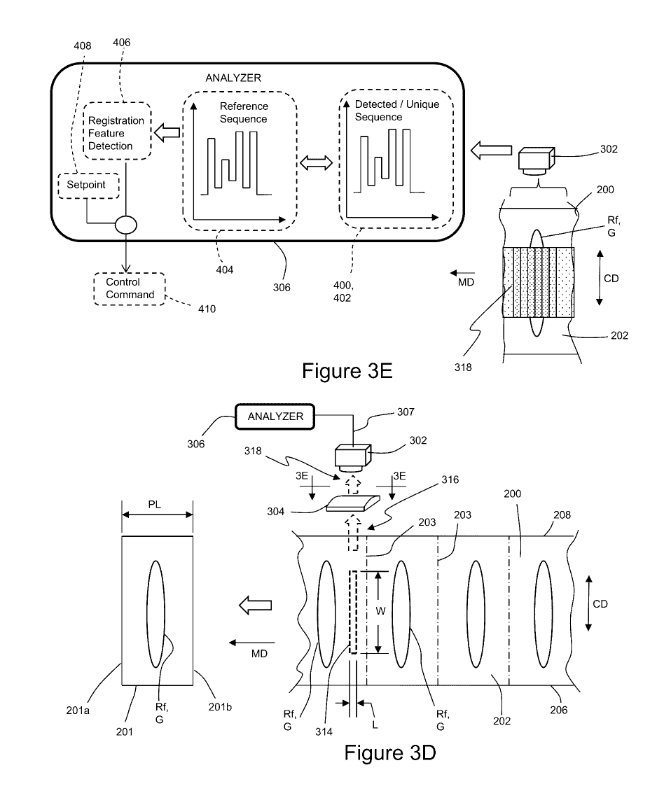

It is to be appreciated that the registration systems and methods disclosed herein are applicable to work with various types of converting processes and/or machines. For example, FIGS. 3A-3D show schematic representations of a converting process including a registration apparatus or system 300 for registering a substrate 200 advancing in a machine direction MD. The substrate 200 may be continuous substrate and include a first surface 202 and an opposing second surface 204, and a first longitudinal side edge 206 and a second longitudinal side edge 208 separated from the first longitudinal side edge 206 in a cross direction CD. It is to be appreciated that the substrate 200 may be subject to additional manufacturing operations, such as combining and/or cutting operations, during assembly of a product. For example, the advancing substrate 200 shown in FIG. 3A may be cut into discrete components 201. The discrete components 201 may have a pitch length PL defined by the distance between a leading edge 201a and a trailing edge 201b. As such, it is to be appreciated that systems and methods herein may be configured to control continuous substrates 200 and/or discrete components 201.

As shown in FIGS. 3A and 3D, the substrate 200 may also include additional objects, such as graphics G. As such, the registration system 300 may be configured to detect registration features Rf on the advancing substrate 200, and in turn, make desired adjustments to the speed and/or tension of the advancing substrate 200 to help ensure the advancing substrate 200 is properly positioned with respect to various manufacturing operations, such as cutting and combining operations. For example, the registration system 300 may utilize the registration features Rf to ensure that the substrate 200 is cut in desired locations such that the graphics G are properly positioned on the discrete components 201 cut from the substrate 200. In another example, the registration system 300 may utilize the registration features Rf to control other assembly operations to ensure that the discrete component 201 is properly positioned in a final product assembly. The registration features Rf and graphics G are generically illustrated as oval shapes in FIG. 3A. As discussed below, the registration features Rf may include all or portions of the graphics G and/or may be separate marks on the substrate.

With continued reference to FIGS. 3A-3D, the registration system 300 may be configured to interact with, monitor, and/or control a converting line. In some configurations, the registration system 300 may include a sensor 302 and a convex cylindrical lens 304 to detect registration features Rf on the advancing substrate 200. The sensor 302 and cylindrical lens 304 may be arranged adjacent the advancing substrate 200, and the sensor 302 may communicate with an analyzer 304. Based on such communications, the analyzer 306 may monitor and affect various operations on the converting line. For example, the analyzer 306 may send various types of control commands to the converting line based on communications with the sensors 302. In some embodiments, the control commands may be the form commands to increase or decrease substrate 200 advancement speeds and/or commands to reposition the substrate 200 in cross direction CD. As shown in FIG. 3D, the analyzer may virtually divide the substrate into virtual components illustrated by virtual lines 203, wherein the spacing between the virtual lines 203 may correspond with the pitch lengths PL of discrete components 201 cut from the substrate 200. As such, control commands may also be used to help ensure the substrate 200 is cut in desired locations.

It is to be appreciated that the analyzer 306 may be configured in various ways. For example, the analyzer 306 may be in the form of a personal computer (PC), a central processing unit (CPU), a field programmable gate array (FPGA), an application specific integrated circuit (ASIC), or a graphical processing unit (GPU). FPGA examples may include the National Instruments PCIe-1473R, National Instruments PXIe-1435, National Instruments 1483R with FlexRIO FPGA module, Altera Stratix II, Altera Cyclone III, Xilinx Spartan 6, Xilink Vertex 6 or Vertex 7. GPU examples may include GeForce GTX 780 (Ti), Quadro K6000, Radeon R9 295X2 and Radeon HD 8990.

It is to be appreciated that the analyzer 306 may also be configured to communicate with one or more computer systems, such as for example, a programmable logic controller (PLC) and/or personal computer (PC) running software and adapted to communicate on an EthernetIP network. Some embodiments may utilize industrial programmable controllers such as the Siemens S7 series, Rockwell ControlLogix, SLC or PLC 5 series, or Mitsubishi Q series. The aforementioned embodiments may use a personal computer or server running a control algorithm such as Rockwell SoftLogix or National Instruments Labview or may be any other device capable of receiving inputs from sensors, performing calculations based on such inputs and generating control actions through servomotor controls, electrical actuators or electro-pneumatic, electrohydraulic, and other actuators. Process and product data may be stored directly in the aforementioned computer systems or may be located in a separate data historian. In some embodiments, the historian is a simple data table in the controller. In other embodiments, the historian may be a relational or simple database. Common historian applications include Rockwell Automation Factory Talk Historian, General Electric Proficy Historian, OSI PI, or any custom historian that may be configured from Oracle, SQL or any of a number of database applications. It is also to be appreciated that the analyzer 306 may be configured to communicate with various types of controllers and inspection sensors configured in various ways and with various algorithms to provide various types of data and perform various functions, for example, such as disclosed in U.S. Pat. Nos. 5,286,543; 5,359,525; 6,801,828; 6,820,022; 7,123,981; 8,145,343; 8,145,344; and 8,244,393; and European Patent No. EP 1528907 B1, all of which are incorporated by reference herein.

It is to be appreciated that various different types of inspection sensors 302 may be used to detect registration features Rf. For example, inspection sensors 302 may be configured as photo-optic sensors that receive either reflected or transmitted light and serve to determine the presence or absence of a specific material. Particular examples of inspection sensors 302 may include simple vision based sensors such as for example: KEYENCE America CZ series RGB fiber optic sensors; SICK CS series sensors, and Banner Engineering QC series color sensors. The sensors may include red, green, blue (RGB) analog outputs that can characterize sensed registration features, such as colors of printed graphics, on advancing substrates by a unique sequence across a specific channel or a combination of channels. For example, some systems may utilize a color sensor, such as a red, green, blue (RGB) color sensor, that may be calibrated or “trained” to detect a specific registration mark color. A suitable such sensing system is available from Keyence of America, Schaumburg, Ill., as the Keyence PS56 System, including suitable transmitter, receiver, and amplifier. In some configurations, the RGB channels may be transformed into alternative orthogonal spaces such as HSL (hue, saturation, luminance).

As shown in FIGS. 3A-3C, the analyzer 306 may be in communication with the sensor 306 through a communication network 307. As such, it is to be appreciated that the analyzer 306 may be physically located near the advancing substrate 200 and/or sensor 302 and/or may be located at another location and in communication with the sensor 302 via a wired and/or wireless network 307. In some embodiments, the communication network 307 is configured as a non-deterministic communication network, such as for example, Ethernet or Ethernet IP (industrial protocol) communication network.

With continued reference to FIGS. 3A-3C, the convex cylindrical lens 304 includes a first surface 308 and an opposing convex surface 310. The cylindrical lens 304 may be in the form of plano convex cylindrical lens wherein the first surface 308 is an elongate flat surface, and wherein the convex surface 310 includes an apex line 312 extending in the machine direction MD. The first surface 308 of the cylindrical lens 304 may be flat. However, it is to be appreciated that in some embodiments, the first surface 308 of the cylindrical lens 304 may be substantially flat, and may also be slightly convex or concave. The convex cylindrical lens 304 is positioned between the substrate 200 and the sensor 302, wherein the first surface 308 of the convex cylindrical lens 304 is in a facing relationship with the first surface 202 of the advancing substrate 200. The relative positions of the sensor 302, the convex cylindrical lens 304, and the substrate 200 establishes a field of view of the sensor 302, referred to herein as a detection zone 314 on the substrate 200. As shown in FIG. 3D, the detection zone defines a length L extending the machine direction MD and width W extending in the cross direction CD, wherein W is greater than L such that the detection zone is elongated in the cross direction CD relative to the machine direction MD. It is to be appreciated that the registration systems 300 herein may be configured with various sizes, shapes, and/or positions of detection zones 314 based on the types of lenses 304 and sensors 302 used as well as the relative positions of the sensors 302, lenses 304, and/or substrate 200.

As shown in FIGS. 3D and 3E, light 316 reflected from detection zone 314 on the first surface 202 of the substrate 200 passes through the convex cylindrical lens 304 and is defocused or blurred. From the convex cylindrical lens 304, the blurred or defocused light 318 is detected by the sensor 302. More particularly, reflected light 316 from the detection zone 314 is blurred or defocused by the convex cylindrical lens 304 along the cross direction CD while maintaining sharpness in the machine direction MD. In turn, the blurring or defocusing creates an averaging effect of hue values of reflected light 316 from the detection zone 314. As such, the defocused light 318 detected by the sensor 302 includes an averaged hue value of the light 316 reflected from the detection zone 314 on the substrate 200. For example, as shown in FIG. 3E, an area of the first surface 202 of the substrate 200 advancing through the detection zone 314 may include a plurality of different colors. The cylindrical convex lens 304 defocuses or blurs the multiple colors of reflected light 316 from the detection zone 314 such that the defocused light 318 detected by the sensor 318 includes a single hue value that corresponds with an average of the hue values from the multiple colors of reflected light 316. It is to be appreciated that the hue values may be determined in various ways, such as for example, by calculations using signals such as RGB analog signals, spectral waveform signals, or LAB color signals.

With reference to FIG. 3A, as the substrate 200 advances in the machine direction MD, the sensor 304 generates signals corresponding with hue values of sensed defocused light 318 reflected from the first surface 202 of the substrate 200 advancing through the detection zone 314. The signals from the sensor 304 are communicated to the analyzer 306 and are transformed into a detected sequence 400 based on the colors of light 316 reflected from the detection zone 314. As shown in FIG. 3A, the detected sequence 400 is schematically represented by a line graph that may be defined by a continuous series of average hue values detected by the sensor 304 across the detection zone 314 at different positions of the substrate 200 along the machine direction MD. It is to be appreciated that the detected sequence 400 shown in FIG. 3A is an example for the purposes of illustrative purposes only, and may not correspond with actual signals from the sensor.

It is to be appreciated that colors on a surface of substrate may be adapted to generate a unique sequence of hue values that may be used with the systems and methods herein to perform registration control of an advancing substrate. For example, as shown in FIGS. 3D and 3E, the first surface 202 of the substrate 200 may include graphics G. And the graphics G may also be defined by a plurality of colors along the cross direction CD and/or machine direction MD. As such, the registration control system 300 may utilize portions or entireties of the graphics G as registration features Rf. As the substrate 200 advances in the machine direction MD, the registration features Rf are advanced through the detection zone 314. As discussed above, light 316 is reflected from the detection zone 314 and through the convex cylindrical lens 304, and the sensor 302 detects defocused or blurred light 318 from the convex cylindrical lens 304. In turn, the sensor 302 generates signals corresponding with hue values of the sensed defocused light 318 and communicates the signals to the analyzer 306. The analyzer 306 transforms the signals received from the sensor 302 into a detected sequence 400. All or a portion of the detected sequence 400 along the machine direction MD of the substrate 200 may be identified as a unique sequence 402 corresponding with the plurality of colors of registration features Rf advancing through the detection zone 314. The analyzer 306 may then compare the unique sequence 402 with a reference sequence 404 as a basis for establishing a detection 406 of a registration feature Rf. It is to be appreciated that the unique sequence 402 and the reference sequence 404 shown in FIG. 3E are examples for the purposes of illustrative purposes only, and may not correspond with actual output signals. Thus, the analyzer 306 may then use the detection 406 of the registration feature Rf as compared with a setpoint 408 to generate control commands 410. As mentioned above, control commands 410 may for example, include changing the speed and/or tension of the advancing substrate 200. As such, the registration system 300 may utilize control commands 410 to control various assembly operations on a converting line for example. For example, control commands 410 may be used to help ensure that the substrate 200 is cut in desired locations to establish desired pitch lengths PL of the discrete components 201 cut from the substrate 200 and/or that the graphics G are properly positioned on such discrete components 201. In other examples, control commands 410 may control other assembly operations to help ensure that discrete components 201 are properly positioned in a final product assembly.

It is to be appreciated that the reference sequence 404 may be created by various signals corresponding to transmitted or reflected light signals other than hue, such as for example, luminance, saturation, emissivity, and delta E.

It is to be appreciated that the reference sequence 404 may be created and stored in the analyzer in various ways. For example, in some configurations, the reference sequence 404 may be created by advancing a substrate with registration features Rf past the cylindrical convex lens 304 and sensor 302 as discussed above in order to generate the unique sequence 402, wherein the unique sequence 402 is stored in the analyzer 306 as a reference sequence. In some configurations, the reference sequence 404 may be created with image processing software and signal analyzer to generate the reference based on an image of a registration feature. In yet another configuration, the reference sequence may be created base on an artwork file as opposed to an image of a registration feature.

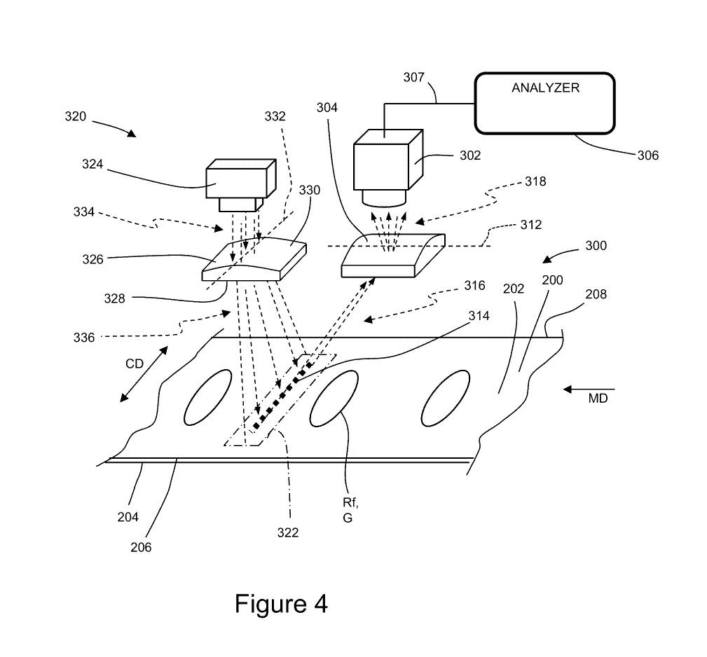

It is to be appreciated that the registration system 300 may also include an illumination apparatus 320 such as shown for example in FIG. 4. More particularly, the illumination apparatus 320 may be configured to define an illumination zone 322 that illuminates the detection zone 314 and a portion of the advancing substrate 200. Thus, as the substrate 200 advances in the machine direction MD, the registration features Rf are advanced through the illumination zone 322 and detection zone 314. And as discussed above, light 316 is reflected from the detection zone 314 and through the convex cylindrical lens 304. And the sensor 302 detects defocused or blurred light 318 from the convex cylindrical lens 304. In turn, the sensor 302 generates signals corresponding with hue values of the sensed defocused light 318 and communicates the signals to the analyzer 306. The analyzer 306 then transforms the signals received from the sensor 302 into detected sequence 400. It is to be appreciated that the outer perimeter or contours of the illumination zone 322 may or may not exactly correspond with the outer perimeter or contours of the detection zone 314. In some embodiments, the illumination zone 322 may illuminate areas of the advancing substrate 200 outside of the detection zone 314. In addition, it is to be appreciated that the inspection system 300 may be configured such that the illumination zone 322 and the detection zone 314 are located on the same surface of the advancing substrate 200 or located on opposing surfaces of the advancing substrate 200. For example, as shown in FIG. 4, the illumination apparatus 320 may be configured to illuminate a portion of the first surface 202 of the substrate 200 advancing through the detection zone 314.

It is to be appreciated that the illumination apparatus 320 may be configured in various ways. For example, as shown in FIG. 4, the illumination apparatus 320 may include a light source 324. In some embodiments, the light source 324 may comprise line lights such as light emitting diode (LED) line lights. Examples of such lights include the ADVANCED ILLUMINATION IL068, various line lights available from METAPHASE, various line lights available from VOLPI such as model number 60023, as well as various line lights available from CCS AMERICA, INC. In some embodiments, the light source 324 may include halogen or other source lights coupled to illuminate the illumination zone 322 and detection zone 314 with fiber bundles and/or panels. Other example light source 324 configurations may include halogen or other sources coupled to fiber bundles. For example, halogen sources may include those available from SCHOTT and fiber bundles and/or panels may include those available from SCHOTT and/or FIBEROPTICS TECHNOLOGY INC. In addition, the light source 324 may be configured to emit light in any suitable frequency range including, for example, ultra-violet, visible and/or infrared.

As shown in FIG. 4, the illumination apparatus 320 may also include a second convex cylindrical lens 326 including a first surface 328 and an opposing convex surface 330. As such, the illumination zone 322 and the detection zone 314 may be illuminated by directing light 334 from the light source 324 through the second convex cylindrical lens 326 such that focused light 336 exiting the second cylindrical convex lens 326 is directed onto the first surface 202 of the continuous substrate 200. The second convex cylindrical lens 326 may be in the form of plano convex cylindrical lens wherein the first surface 328 is an elongate flat surface, and wherein the convex surface 330 includes an apex line 332 extending in the cross direction CD. The first surface 328 of the cylindrical lens 326 may be flat. However, it is to be appreciated that in some embodiments, the first surface 328 of the cylindrical lens 326 may be substantially flat, and may also be slightly convex or concave. The second convex cylindrical lens 326 is positioned between the substrate 200 and the light source 324, wherein the first surface 328 of the second convex cylindrical lens 326 is in a facing relationship with the first surface 202 of the advancing substrate 200. It is to be appreciated that the registration systems 300 herein may be configured with various sizes, shapes, and/or positions of illumination zones 322 based on the types of lenses 326 and light sources 324 used as well as the relative positions of the light sources 324, lenses 326, and/or substrate 200.

To provide additional context to the above discussion of the registration system configurations of FIGS. 3A-3E and 4, the following provides a description of example implementations of the registrations systems and processes.

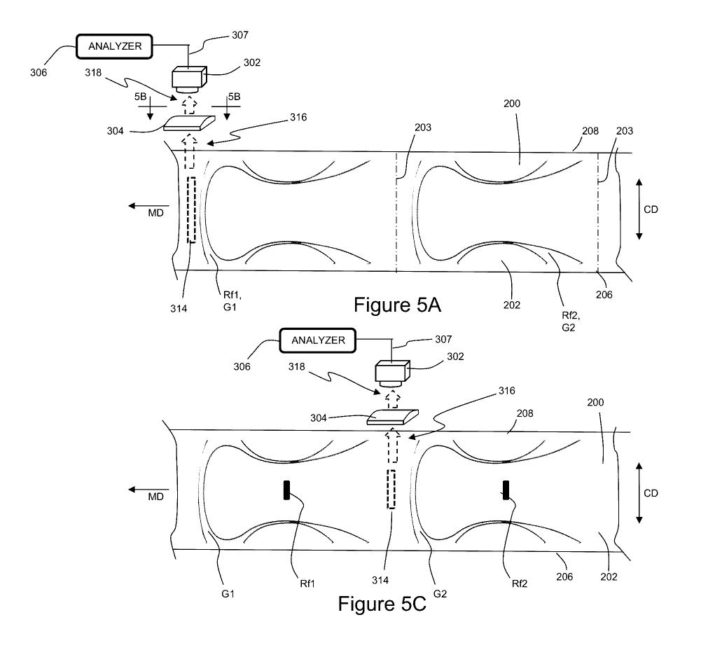

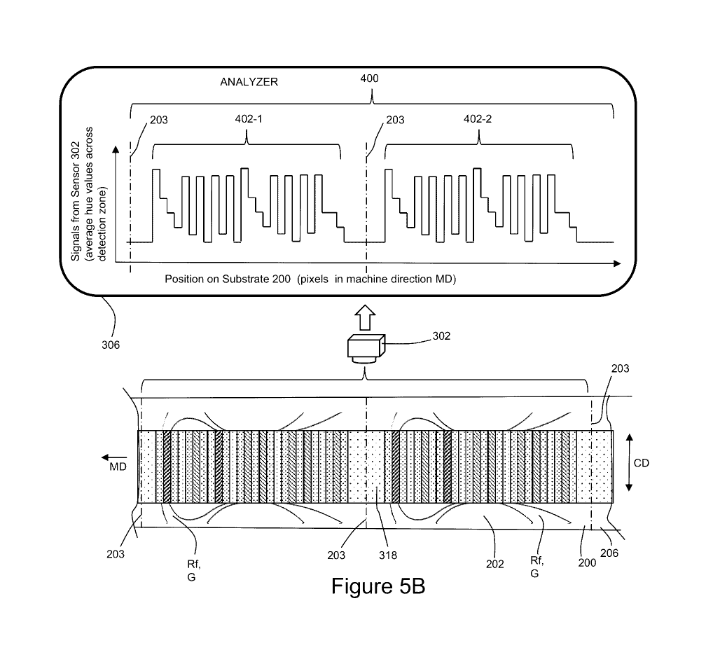

For example, FIG. 5A is a detailed view of a continuous substrate 200 advancing in a machine direction MD. The substrate 200 also includes a plurality of graphics G1, G2 positioned along the length of the first surface 202 of the substrate 200. Although only graphics G1 and G2 are illustrated, it is to be appreciated that the substrate 200 may include several graphics G positioned along the machined direction MD of the substrate 200. It is to be appreciated that the graphics G1, G2 may also be utilized a registration features Rf1, Rf2 as discussed above. More particularly, as the substrate 200 advances in the machine direction MD, the graphics G1, G2 advance through the detection zone 314. And light 316 is reflected from the detection zone 314 and through the convex cylindrical lens 304, and the sensor 302 detects defocused or blurred light 318 from the convex cylindrical lens 304. In turn, the sensor 302 generates signals corresponding with hue values of the sensed defocused light 318 and communicates the signals to the analyzer 306. As discussed above, the analyzer 306 transforms the signals received from the sensor 304 into a detected sequence 400 based on the colors of light 316 reflected from the detection zone 314.

As shown in FIG. 5B, the detected sequence 400 is schematically represented by a line graph defined by a continuous series of average hue values detected by the sensor 304 across the detection zone 314 at different positions of the substrate 200 along the machine direction MD. It is to be appreciated that the detected sequences 400 shown in FIG. 5B is an example for the purposes of illustrative purposes only, and may not correspond with actual output signals. In addition, all or a portion of the detected sequence 400 along the machine direction MD of the substrate 200 may be identified as a unique sequence 402 corresponding with the plurality of colors of registration features Rf advancing through the detection zone 314. For example, as shown in FIG. 5B, a portion of the first graphic G1 advancing through the detection zone 314 corresponds with a first registration feature Rf1, and a portion of the second graphic G2 advancing through the detection zone 314 corresponds with a second registration feature Rf2. In turn, the first unique sequence 402-1 corresponds with the first registration Rf1, and the second unique sequence 402-2 corresponds with the second registration Rf2. As shown in the example in FIG. 5B, where graphics G1 and G2 have the same colors and shapes, the unique sequences 402-1 and 400-2 may also be the same. As discussed above with reference to FIG. 3E, the analyzer 306 may then compare the unique sequence 402 with a reference sequence 404 as a basis for establishing detections 406 of registration features Rf1 and Rf2. In an example where the registration system 300 is configured to monitor and control a substrate 200 with identical graphics G, the analyzer may use only one reference sequence.

It is to be appreciated that the advancing substrate 200 shown in FIGS. 5A and 5B may be utilized in various converting operations. For example, the substrate 200 may be advanced through a diaper assembly process and converted into a backsheet 138 such as discussed above with reference to FIGS. 1A-1C. As such, the substrate 200 is illustrated in FIGS. 5A and 5B with virtual lines 203, wherein the spacing between the virtual lines 203 may correspond with the pitch lengths PL of discrete backsheets 138 cut from the substrate 200. As such, control commands may also be used to help ensure the substrate 200 is cut in desired locations to provide desired lengths of backsheets 138.

It also to be appreciated that the registration systems and methods herein may also be configured to monitor and control substrates 200 having various configurations, shapes, and/or designs of graphics G, wherein portions or entireties of such graphics are utilized as registration features. It is also to be appreciated that the registration systems and methods herein may also be configured to monitor and control substrates 200 with separated marks or graphics designated as registration features Rf. For example, FIG. 5B is a detailed view of the continuous substrate 200 of FIG. 5A also including distinct registration features Rf1 and Rf2. The registration system 300 may be adapted to detect the distinct registration features Rf1 and Rf2 such as shown in FIG. 5C alone or in combination with other graphics G1, G2. It is to be appreciated that such distinct registration features may be utilized in converting processes wherein the registration features are included in final product assemblies or are removed from the substrate prior to final product assembly.

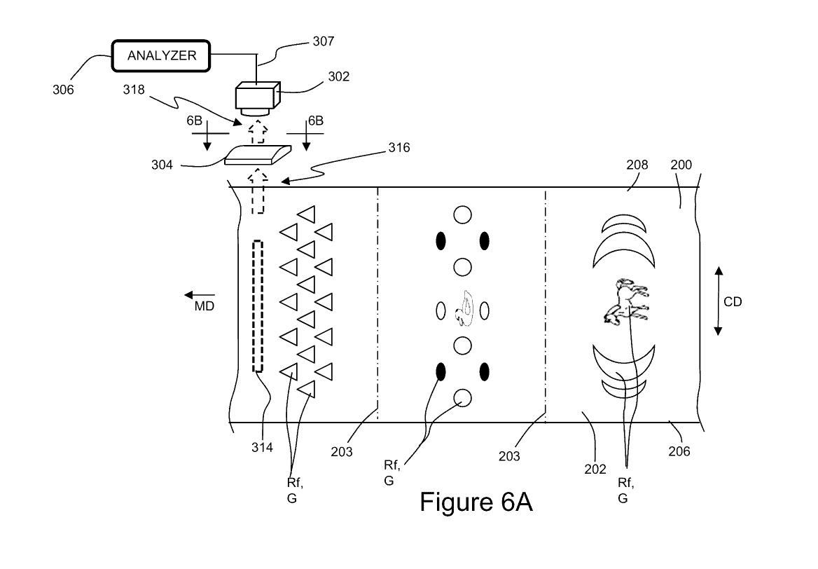

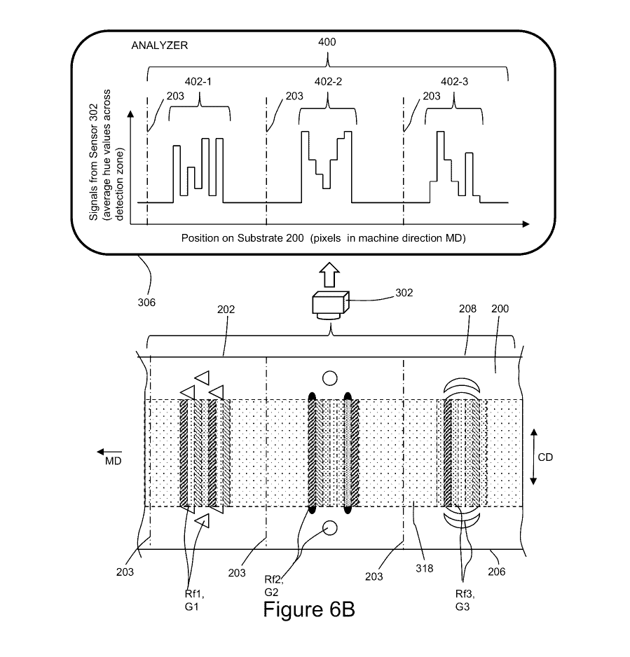

FIG. 6A shows another detailed view of a continuous substrate 200 advancing in a machine direction MD and including graphics G of different shapes and/or colors. Although only graphics G1, G2, and G3 are illustrated, it is to be appreciated that the substrate 200 may include several graphics G positioned along the machined direction MD of the substrate 200.

The graphics G1, G2, G3 may also be utilized a registration features Rf1, Rf2, Rf3 as discussed above. As discussed above, as the substrate 200 advances in the machine direction MD, the graphics G1, G2, G3 advance through the detection zone 314, and the analyzer 306 transforms the signals received from the sensor 304 into a detected sequence 400 based on the colors of light 316 reflected from the detection zone 314. As shown in FIG. 6B, a portion of the first graphic G1 advancing through the detection zone 314 corresponds with a first registration feature Rf1; a portion of the second graphic G2 advancing through the detection zone 314 corresponds with a second registration feature Rf2; and a portion of the third graphic G3 advancing through the detection zone 314 corresponds with a third registration feature Rf3. In turn, the first unique sequence 402-1 corresponds with the first registration Rf1; the second unique sequence 402-2 corresponds with the second registration Rf2; and the third unique sequence 402-3 corresponds with the third registration Rf3. As shown in the example in FIG. 6B, where graphics G1, G2, and G3 have different colors and/or shapes, the unique sequences 402-1, 400-2, and 400-3 may also be different. It is to be appreciated that the detected sequences 400 shown in FIG. 6B is an example for the purposes of illustrative purposes only, and may not correspond with actual output signals. In an example where the registration system 300 is configured to monitor and control a substrate 200 with different graphics G corresponding with different registration features having different unique sequences, the analyzer may use different reference sequences to establish detections of the different registration features Rf.

It is to be appreciated that the advancing substrate 200 shown in FIGS. 6A and 6B may be utilized in various converting operations. For example, the substrate 200 may be advanced through a diaper assembly process and converted into a connection zone 168 such as discussed above with reference to FIGS. 1A-1C. As such, the substrate 200 is illustrated in FIGS. 6A and 6B with virtual lines 203, wherein the spacing between the virtual lines 203 may correspond with the pitch lengths PL of discrete connection zones 168 cut from the substrate 200. As such, control commands may also be used to help ensure the substrate 200 is cut in desired locations.

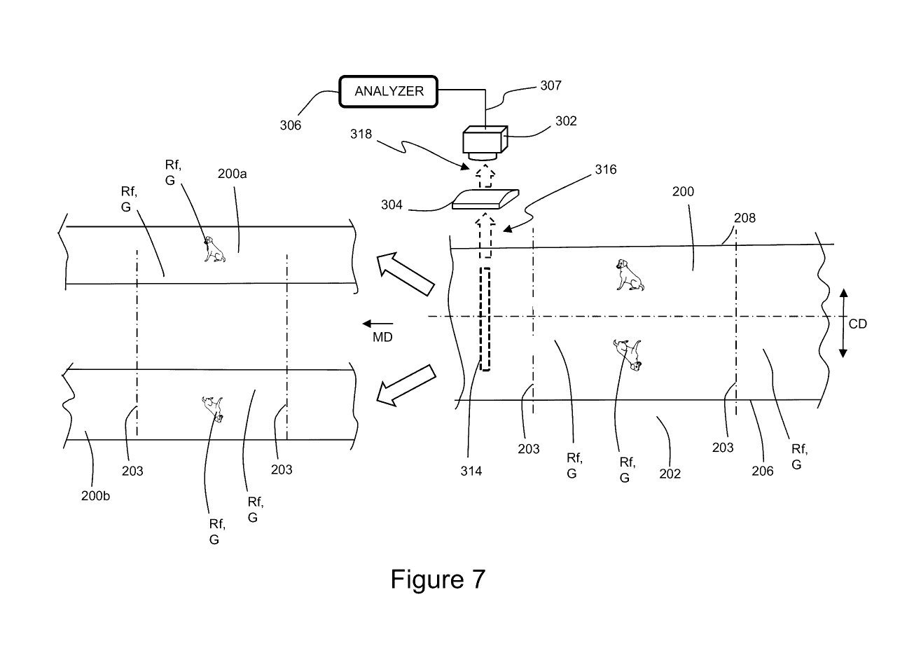

In yet another example, the registration system 300 herein may be configured to utilize graphics on substrates as registration features, wherein the substrates are subsequently converted into different components of an assembled article. For example, FIG. 7 shows an advancing substrate 200 with various graphics G advancing through a diaper converting process, wherein the graphics G may be used as registration features Rf. The substrate 200 may be subsequently slit along the machine direction MD into first and second substrates 200a, 200b, each including graphics G. In turn, the substrates 200a, 200b may be converted into front and rear belts 172, 174, such as discussed above with reference to FIGS. 2A-2C. As such, the substrate 200 is illustrated in FIG. 7 with virtual lines 203, wherein the spacing between the virtual lines 203 may correspond with the pitch lengths PL of front and rear belts 172, 174 cut from the substrate 200.

It is to be appreciated that the registration feature detection systems and methods disclosed herein may be adapted to work with various types of registrations systems and converting processes and/or machines, such as those disclosed for example in U.S. Pat. Nos. 5,795,280; 5,818,719; 5,930,139; 6,068,362; 6,352,497; 6,354,984; 6,444,064; 6,649,808; 6,652,686; 6,764,563; 6,869,386; 6,957,160; 6,955,733; 7,082,347; 7,123,981; 8,145,344; 8,145,343; 8,157,776; and 8,168,254; as well as U.S. Patent Publication Nos. 2003/0233081 A1 and 2015/0250655 A1; and PCT Publication No. WO 02/03900 A1.

This application claims the benefit of U.S. Provisional Application No. 62/253,710 filed on Nov. 11, 2015, the entirety of which is incorporated herein by reference.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for assembling disposable absorbent articles, the method comprising the steps of:
providing a continuous substrate extending in a machine direction and defining a width in a cross direction, the continuous substrate comprising a first surface and an opposing second surface, the continuous substrate further comprising registration features;
providing a sensor;
establishing a detection zone on the first surface of the substrate by positioning a convex cylindrical lens between the first surface of the substrate and the sensor, the convex cylindrical lens comprising a first surface and an opposing convex surface, the convex surface comprising an apex line extending in the machine direction, wherein the first surface of the convex cylindrical lens is in a facing relationship with the first surface of the substrate, and wherein the detection zone defines a length extending the machine direction and width extending in the cross direction such that the detection zone is elongated in the cross direction relative to the machine direction;
advancing the registration features through the detection zone by advancing the substrate in the machine direction at a first speed;
defocusing light reflected from the detection zone through the convex cylindrical lens;
detecting the defocused light passing from the convex cylindrical lens with the sensor; and
generating signals corresponding with hue values of detected defocused light reflected from the registration features advancing through the detection zone.

2. The method of claim 1, further comprising the step of:
providing a second convex cylindrical lens comprising a first surface and an opposing convex surface, the convex surface comprising an apex line extending in the cross direction, wherein the first surface of the convex cylindrical lens is in a facing relationship with the first surface of the advancing substrate and wherein the convex surface is in a facing relationship with a light source; and
further comprising the step of illuminating the detection zone by directing light from the light source through the second convex cylindrical lens and onto the first surface of the continuous substrate.

3. The method of claim 1, wherein the first surface of the convex cylindrical lens is substantially flat.

4. The method of claim 1, further comprising the step of communicating the signals corresponding with hue values to an analyzer selected from the group consisting of: a field programmable gate array, an application specific integrated circuit, and graphical processing unit

5. The method of claim 1, wherein the registration features comprise printed graphics.

6. The method of claim 5, wherein the printed graphics comprise a plurality of colors.

7. The method of claim 1, wherein the registration features comprise registration marks.

8. The method of claim 1, further comprising the step of cutting the continuous length of absorbent articles into discrete components of an assembled absorbent article.

9. The method of claim 8, wherein lengths of the discrete components correspond to pitch lengths of individual absorbent articles.

10. The method of claim 9, wherein the discrete components comprise backsheets.

11. The method of claim 8, wherein the discrete components comprise connection zones.

12. The method of claim 8, wherein the discrete components comprise belts.

13. The method of claim 1, further comprising the step of transforming the signals into a unique sequence based on a color pattern of the registration feature.

14. The method of claim 13, further comprising the step of: comparing the unique sequence to a reference sequence.

15. The method of claim 14, further comprising the step of changing the first speed to a second speed based on the comparison of the unique sequence to the reference sequence.

16. The method of claim 1, wherein the hue values are determined though calculations based at least one signal from the group consisting of: RBG analog signals, spectral waveform signals, and LAB color signals.

17. A method for assembling disposable absorbent articles, the method comprising the steps of:
providing a continuous substrate extending in a machine direction and defining a width in a cross direction, the continuous substrate comprising a first surface and an opposing second surface, the continuous substrate further comprising registration features;
illuminating an elongate illumination zone extending in the cross direction on the first surface of the advancing substrate;
providing a convex cylindrical lens comprising a first surface and an opposing convex surface, the convex surface comprising an apex line extending in the machine direction, wherein the first surface of the convex cylindrical lens is in a facing relationship with the first surface of the advancing substrate;
advancing the registration features through the illumination zone by advancing the substrate in the machine direction at a first speed;
defocusing light reflected from the elongate illumination zone through the convex cylindrical lens;
sensing the defocused light passing from the convex cylindrical lens; and
generating signals corresponding with hue values of sensed defocused light reflected from the registration features advancing through the illumination zone.

18. The method of claim 17, further comprising the step of:
providing a second convex cylindrical lens comprising a first surface and an opposing convex surface, the second convex surface comprising an apex line extending in the cross direction, wherein the first surface of the second convex cylindrical lens is in a facing relationship with the first surface of the advancing substrate and wherein the second convex surface is in a facing relationship with a light source; and
wherein the step of illuminating the illumination zone further comprises directing light from the light source through the second convex cylindrical lens and onto the first surface of the continuous substrate.

19. The method of claim 17, wherein the first surface of the convex cylindrical lens is substantially flat.

20. The method of claim 17, wherein the registration features comprise multicolored printed graphics.

21. The method of claim 17, further comprising the step of transforming the signals into a unique sequence based on a color pattern of the registration feature.

22. The method of claim 21, further comprising the step of: comparing the unique sequence to a reference sequence.

23. The method of claim 17, wherein the hue values are determined though calculations based on at least one signal from the group consisting of: RBG analog signals, spectral waveform signals, and LAB color signals.

24. An absorbent article manufacturing apparatus for controlling the speed of a continuous substrate, the continuous substrate extending in a machine direction and defining a width in a cross direction, the continuous substrate comprising a first surface and an opposing second surface, the continuous substrate further comprising registration features, the apparatus comprising:
a convex cylindrical lens comprising a first surface and an opposing convex surface, the convex surface comprising an apex line extending in the machine direction, wherein the first surface of the convex cylindrical lens is in a facing relationship with the first surface of the substrate, and defining a detection zone having a length L extending the machine direction and width W extending in the cross direction, wherein W is greater than L such that the detection zone is elongated in the cross direction relative to the machine direction;
a sensor positioned adjacent the convex cylindrical lens to define a detection zone on the first surface of the substrate and having a length L extending the machine direction and width W extending in the cross direction, wherein W is greater than L such that the detection zone is elongated in the cross direction relative to the machine direction, the sensor configured to generate signals corresponding with hue values of detected defocused light from the convex cylindrical lens as reflected from the registration features advancing through the detection zone; and
an analyzer selected from the group consisting of: a field programmable gate array, an application specific integrated circuit, and graphical processing unit, the analyzer configured to transform the signals from the sensor into a unique sequence and to adjust the speed of the substrate based on a comparison of the unique sequence to a reference sequence.

25. The apparatus of claim 24, further comprising:
a light source; and
a second convex cylindrical lens comprising a first surface and an opposing convex surface, the convex surface comprising an apex line extending in the cross direction, wherein the first surface of the convex cylindrical lens is in a facing relationship with the first surface of the advancing substrate and wherein the convex surface is in a facing relationship with the light source.

26. The apparatus of claim 24, wherein the first surface of the convex cylindrical lens is substantially flat.
